(19) United States
(12) Patent Application Publication
(10) Pub. No.: US 2006/0111719 A1
(43) Pub. Date: May 25, 2006

(54) Oval pin for fixing an implant subjected to tensile load

(76) Inventors: Michael J. Strobel, Straubing (DE); Andreas Weiler, Berlin (DE); Wolf Petersen, Munster (DE); Sascha Berberich, Tuttlingen (DE)

Correspondence Address: ST. ONGE STEWARD JOHNSTON & REENS, LLC, 986 BEDFORD STREET, STAMFORD, CT, 06905-5619, US

(21) Appl. No.: 11/266,722

(22) Filed: Nov. 3, 2005

(30) Foreign Application Priority Data
Nov. 3, 2004 (DE) 10 2004 053 464.0

(51) Int. Cl. A61B 17/58

(52) U.S. Cl. 606/72

(57) ABSTRACT

A pin for fixing an implant subjected to tensile load, in particular a tendon implant, has a rod-shaped body, the force exerted by the implant being able to be input onto the cross section of the body, on one side, and being able to be output on the opposite side to an anchoring site. It is proposed that the cross section has a longer dimension, in the direction of the force input from the tendon implant than it has transverse to the longitudinal axis.

### PRIOR APPLICATIONS

This application claims priority of German Patent Application No. 10 2004 053 464.0 filed on Nov. 3, 2004.

### FIELD OF THE INVENTION

The invention relates to a pin for fixing an implant subjected to tensile load, in particular a tendon implant, said pin having a rod-shaped body, a force exerted by the implant being able to be input onto a cross section of the pin, on one side, and being able to be output on an opposite side to an anchoring site.

### BACKGROUND OF THE INVENTION

Pins of this kind are used to fix an implant fitted in an opening in a bone. To do this, the implant is first pushed into the opening, in most cases a bore that has been formed from the outside. The pin is then driven in transversely thereto, thus passing transversely through the bore and through the implant pushed into the latter, as a result of which said implant is fixed in position. Since the pin extends transversely with respect to the longitudinal extent of the implant, the expression “cross pin” has become established.

A common application is in fixing an implant serving as a replacement for the cruciate ligaments of the knee.

For replacing the cruciate ligament, an operating technique has been developed in which the implant or graft, in most cases a tendon from the patient, is formed into a loop, and the pin is driven transversely through it in the area of the loop. It is also known for two loose ends of tendon sections to be sewn together. When moving the knee a strong tension acts on the tendon implant which is transferred to the pin.

The tendon loops round the rod-shaped body of the pin in a cross-sectional plane. The result of this is that the tensile load exerted by the tendon is input into the rod-shaped body on one side of its cross section and can be output on the opposite side to anchoring sites which are axially spaced apart from the input side. This anchoring site is the inner wall, geometrically speaking a surface line of the wall, of a bore in which the pin is received.

Such pins usually have a circular cross section so that the force output site lies along a surface line of the rod-shaped body lying opposite the loop or cross-section side around which the loop is guided. Additionally, the force input side is located in the central section of the pin, the force output sides are located at the opposite end sections of the pin resting in the bone.

In the event of loading, this has the effect that the force input by the tendon implant is output to the anchoring site in a more or less limited area on the end sections of the pin.

A study has established that this geometry can cause the pin to fracture.

In order to remedy this situation, it was attempted to introduce a second identical pin, with a likewise circular cross section, directly below the first pin in the direction of the force input.

A further study has now shown that even the placement of a second adjacent pin is unable to exclude the possibility of fracturing. On the contrary, if the first pin fractures under strong tensile loading, then the second pin also fractures thereafter. There is, as it were, a kind of domino effect.

It is therefore an object of the present invention is to remedy this situation and make available a pin of the aforementioned type for fixing an implant subjected to tensile load, which pin is able to withstand high tensile loads without fracturing.

### SUMMARY OF THE INVENTION

According to the invention, this object is achieved by the fact that the cross section has a longer dimension, in the direction of the force input from the tendon implant, than it has transverse to the longitudinal direction.

It was found that greater safety against fracturing can be achieved by means of a deviation from a round geometry and by arranging the pin in such a way that the longer cross-sectional dimension extends in the direction of the force input from the tendon implant.

A further advantage was found to be that the tendon engages around the implant about a relatively large circumference angle and across a large circumference surface area, resulting in planar contact of the tendon implant in the bore, which promotes incorporation of the tendon. The tendon is also more strongly compressed than by a round pin, resulting in better fixing.

In a further embodiment of the invention, the cross section of the pin is oval.

It was found that particularly favorable resistance to fracturing can be achieved with this geometry and that the tendon implant can bear intimately on the pin within a large area, thus promoting incorporation, which in the end also contributes to the resistance to fracturing, since a tendon firmly fused to the area surrounding the bone can divert some of the load via these growth points.

In a further embodiment, the cross section is configured as a flat oval.

Not only is this easy to obtain from the manufacturing point of view, this geometry corresponds approximately to the contour curve of two pins of circular cross section lying next to one another. This means that there does not have to be any more space than in the aforementioned operating techniques with two adjoining pins. However, a much greater resistance to fracturing is achieved.

This does not mean, however, that a pin with an oval cross section now necessarily has to have the size of an orbit of two conventional pins of circular cross section. It can be enough that the longer vertical axis of the cross-sectional profile corresponds to the diameter of a conventional pin.

The important thing is that there is a longer dimension in the direction of the force input, it being possible to use not only oval shapes curved concavely outward, but also bone shapes or other geometries.

It will be appreciated that the aforementioned features and those still to be discussed below can be used not only in the respectively cited combination, but also in other combinations, or singly, without departing from the scope of the present invention.

### BRIEF DESCRIPTION OF THE DRAWINGS

The invention is described and explained in more detail below using a number of illustrative embodiments and with reference to the attached drawings, in which:

FIG. 1 shows a pin in a side view;

FIG. 2 shows a cross section along the line II-II in FIG. 1;

FIG. 3 shows, on a slightly enlarged scale, a cross section, corresponding to FIG. 2, of another illustrative embodiment of oval shape, the figure also indicating a tendon implant which is to be fixed by the pin, and

FIG. 4 shows a highly schematic representation of a pin from FIG. 1 which is fitted in a femur for transfixing an implant subjected to tensile load.

### DETAILED DESCRIPTION OF THE INVENTION

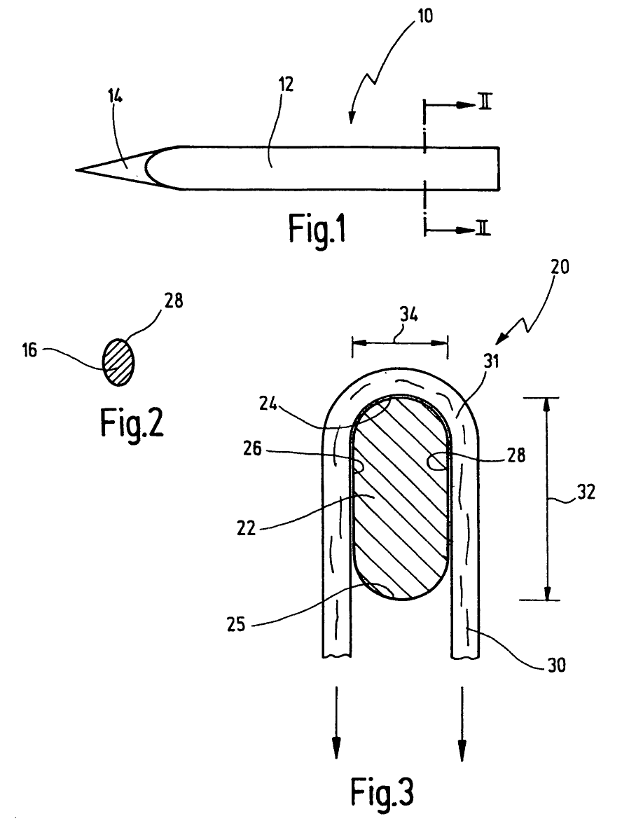

A pin 10 shown in the FIGS. 1 and 2 has a rod-shaped body 12 which, at one end, merges into a conically tapering tip 14.

It will be seen from the view in FIG. 2 that the cross section has an oval shape.

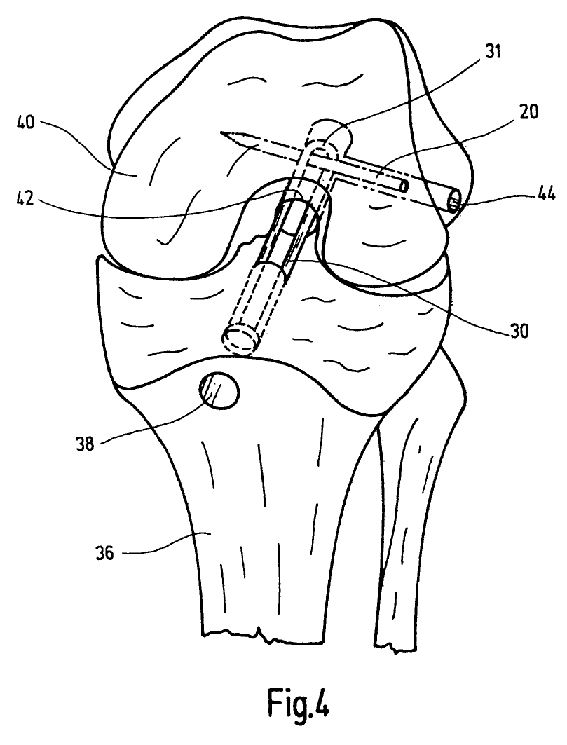

FIG. 3 shows a pin 20 which likewise has a rod-shaped body (not shown here) with a tip, and which has a cross section which is designed as a flat oval 22.

At its opposite ends, the flat oval 22 is delimited by two semicircles 24 and 25 which are connected via two mutually parallel side flanks 26 and 28.

The figure also shows how a tendon implant 30 is placed around the pin 20, specifically in the form of a loop 31.

The pin 20 is oriented in such a way that its long axis 32 is directed upward, that is to say in the direction of the force input by the tendon implant. The short axis 34 extends transverse thereto and also transverse to the longitudinal axis of the pin 20.

The pins have, for example, a length of ca. 40 mm and a diameter of approximately 4.5 mm.

They can be made from absorbable materials, from plastic material or from metal, in particular titanium or a titanium alloy.

FIG. 4 shows a situation in which the pin 20 is used for fixation of a tendon implant 30 serving as a replacement for the cruciate ligament of a human knee.

For this purpose, a through-bore 38 has been formed in the tibia 36 and continues as a blind bore 42 in the femur 40.

A transverse bore 44 is also formed in the femur 40 to allow the pin 20 to be driven in, specifically in such a way that it extends through the loop 32 of the tendon implant 30, which was previously inserted into the blind bore 42.

The operating technique and the necessary auxiliary instruments for forming the bores 38, 42 and for correctly forming the transverse bore 44 and for fitting and securing the implant and the pin are described in particular in US 2003/0065391 A1 and in U.S. Pat. No. 5,601,562, express reference being made to the content of these documents.

If a pin of oval cross section is to be fitted, a drill is first used to form the transverse bore 44 with a round cross section, and a dilator with approximately the same contour as the pin 20 is then driven in. The dilator is in this case slightly smaller than the pin 20 to ensure that a press fit is obtained when the pin 20 is introduced. The oval pin is in each case introduced as shown in FIG. 3, that is to say in such a way that its longer cross-sectional axis 32 extends in the direction of the force input or tensioning direction of the tendon implant 30.

As can be seen from FIG. 4, pin 20 rests in transverse bore 44 extending transversely to blind bore 42. A surface line of transverse bore 44 onto which the rod-shaped body of pin 20 rests represents the anchoring site of pin 20. The tensile load exerted onto pin 20 by the tendon implant 30 in a direction of through-bore 38 in the tibia 36 is distributed into the wall of the transverse bore 44.

In the embodiment of FIG. 4 transverse bore 44 extends from the outside of femur 40 up to blind bore 42. When inserting the pin 20 into transverse bore 44 with a respective tool, the pin 20 is driven beyond the blind bore 42 into the bone material of femur 40 like a nail. It is also possible to provide transverse bore 44 in that it extends beyond the blind bore 42.

1-7. (canceled)

8. A robot comprising:
a fork having a left prong and a right prong:
a left capacitive sensor assembly coupled to the left prong to detect via a change in capacitance when the left prong has come into contact with a barrier as a result of undesired fork movement; and,
a right capacitive sensor assembly coupled to the right prong to detect via a change in capacitance when the right prong has come into contact with a barrier as a result of undesired fork movement.

9. The robot of claim 8, further comprising:
a robot arm coupled to the fork;
a motor to move the robot arm and correspondingly move the fork;
a motor controller communicatively coupled to the left capacitive sensor and the right capacitive sensor, the controller causing the motor to move the robot arm and correspondingly move the fork, the controller stopping movement of the motor and correspondingly stopping movement of the fork in response to the change in capacitance detected by at least one of the left capacitive sensor assembly and the right capacitive sensor assembly.

10. The robot of claim 8, wherein each of the left and the right capacitive sensor assemblies comprises:
a hit contact attached to one of the left and the right prongs and that comes into contact with a barrier as a result of undesired movement by the robot, the hit contact freely moveable along an axis of movement, and moving in one direction along the axis of movement when coming into contact with the barrier and moving in an opposite direction along the axis of movement when backing away after contact with the barrier;
one or more fixed contacts;
one or more damping springs having first ends connected to the hit contact and second ends connected to the one or more fixed contacts, the one or more damping springs being compressed against the one or more fixed contacts when the hit contact comes into contact with the barrier; and,
a capacitive sensor operatively coupled to the hit contact and having a capacitance that changes as the hit contact moves along the axis of movement resulting from coming into contact with the barrier or backing away after contact with the barrier, the capacitance of the capacitive sensor used to detect the one of the left and the right prongs coming into contact with the barrier so that damage to the robot can be prevented.

11. The robot of claim 8, wherein the capacitive sensor of each of the left and the right capacitive sensor assemblies comprises:
a compressible dielectric having a first side and a second side;
a fixed capacitive plate located to the first side of the dielectric;
a movable capacitive plate located to the second side of the dielectric and that can compress the compressible dielectric to change the capacitance of the capacitive sensor; and,
an internal hit plate operatively coupled to the hit contact and located to a side of the movable capacitive plate opposite of another side of the movable capacitive plate on which the second side of the dielectric is located, such that movement of the hit contact results in movement of the internal hit plate, causing the internal hit plate to press against and move the movable capacitive plate, compressing the compressible dielectric and changing the capacitance of the capacitive sensor.

12. The robot of claim 8, wherein the capacitive sensor of each of the left and the right capacitive sensor assemblies comprises:
an internal hit plate operatively coupled to the hit contact;
a dielectric at least partially compressible and having a first side and a second side in-between which the internal hit plate is located, such that movement of the hit contact results in movement of the internal hit plate and compression of the dielectric, changing the capacitance of the capacitive sensor;
one or more first capacitive plates located to the first side of the dielectric; and,
one or more second capacitive plates located to the second side of the dielectric.

13. The robot of claim 8, wherein the robot is used in conjunction with semiconductor device fabrication.

14. The robot of claim 13, wherein the robot is used to transfer semiconductor wafers via the fork, such that the left and the right capacitive sensor assemblies are for preventing damage to the semiconductor wafers.

15-20. (canceled)
